(19) United States
(12) Patent Application Publication
(10) Pub. No.: US 2019/0296424 A1
(43) Pub. Date: Sep. 26, 2019

(54) ANTENNA COUPLING OF IMPLANTABLE DEVICES OR DEVICES ATTACHED TO A PERSON AND AN EXTERNAL DEVICE BY ORIENTATION DEPENDENT SWITCHING OF ANTENNAS

(71) Applicant: BIOTRONIK SE & Co. KG, Berlin (DE)

(72) Inventors: Jens Ulmer, Erlenbach (CH); Thomas Finnberg, Zuerich (CH)

(21) Appl. No.: 16/285,753

(22) Filed: Feb. 26, 2019

(30) Foreign Application Priority Data
Mar. 20, 2018 (EP) 18162738.1

(51) Int. Cl. H01Q 1/27; H04B 7/10; H04B 7/08; H04B 7/06; A61B 5/00

(52) U.S. Cl. CPC H01Q 1/273; H04B 7/10; H04B 7/0834; A61B 5/6823; A61B 5/0031; A61B 5/6867; H04B 7/0608

(57) ABSTRACT

The present disclosure relates to a system, comprising: a first device having at least two antennas for transmitting and/or receiving a radio signal, wherein the first device is a medical device configured to be implanted into a patient, wherein the first device comprises a sensor that is configured to generate an output signal indicative of a spatial orientation of the first device, and wherein the first device is configured to use the output signal to select one of said antennas or to control said antennas for receiving a radio signal and/or for transmitting a radio signal, and a second device having at least one antenna, wherein the second device is configured to transmit a radio signal to the first device and/or to receive a radio signal from the first device, wherein the second device is an external device.

### CROSS-REFERENCE TO RELATED APPLICATIONS

This patent application claims the benefit of and priority to co-pending European Patent Application No. EP 18162738.1, filed on Mar. 20, 2018 in the European Patent Office, which is hereby incorporated by reference in its entirety.

### TECHNICAL FIELD

The present disclosure relates to a system, particularly a medical system, comprising a first device having at least two antennas for transmitting and/or receiving a radio signal.

### BACKGROUND

For transmitters/receivers that are either implanted or worn close to the body, the transmission and reception properties change depending on the position of the patient wearing the transmitter/receiver in relation to an external radio device that interacts with the transmitter/receiver on the person.

International Publication No. WO 2015/196164 describes multiples antennas for an implant, wherein the antennas are arranged differently to make the link gain less sensible to the relative alignment to an external transceiver.

U.S. Publication No. 2013/0095875 discloses antenna apparatuses and related antenna units that include antenna selection based on orientation for a wireless network. Antenna selection is provided between two or more antennas disposed in different polarization orientations according to orientation of the antenna unit in which the antennas are included. The antenna(s) oriented most closely to perpendicular to the ground in one embodiment may be selected for use in wireless communications with wireless client devices. In this manner, the antenna(s) employed in wireless communications is likely to be the closest in polarization to the polarization of wireless client device antennas. Otherwise, an unacceptable reduction in communications link quality with the wireless client devices may occur.

U.S. Publication No. 2016/0174842 discloses an epidermal electronics device, including a barrier layer configured to attach the epidermal electronics device to the skin of a user, an antenna array coupled to the barrier layer, and a control circuit coupled to the actively phased antenna, wherein the control circuit is configured to actively phase the antenna array.

The present invention is directed at overcoming one or more of the above-mentioned problems.

### SUMMARY

Based on the above, it may be an objective to provide an improved radio communication between a first device and a second device that is less sensitive to the orientation of the first device with respect to the second device.

A system having the features of claim 1 is disclosed.

The first device comprises a sensor that is configured to generate an output signal indicative of a spatial orientation of the first device (e.g., with respect to a reference direction or with respect to a second device), wherein the first device is configured to use the output signal to select one of said antennas or to control said antennas for receiving a radio signal (e.g., from the second device of the system) and/or for transmitting a radio signal (e.g., to said second device). The system may comprise a second device (which may have at least one antenna) configured to transmit a radio signal to the first device and/or to receive a radio signal from the first device. The first device may be a medical device configured to be implanted into a patient or to be arranged on a patient. The second device may be an external device that can, e.g., be configured to receive radio signals from the first device or to transmit radio signals to the first device. Particularly, the notion external in this regard means that the second device is arranged in a surrounding of the patient outside the patient, particularly remote from the patient.

Particularly, the disclosure thus allows monitoring a patient, for example, at the sickbed, via a wireless connection that functions reliably. This is advantageous since the body of a patient tends to partly absorb or reflect high-frequency signals in certain positions of the patient. Particularly, the switching of antennas depending on spatial orientation allows to improve antenna coupling and to maintain a functioning communication.

Particularly, the spatial orientation of the first device (and particularly of the associated antennas) is defined by three parameters corresponding to the three degrees of freedom of the spatial orientation. The parameters can be three angles such as azimuth, pitch and roll.

Furthermore, according to an embodiment of the system, the first device is configured to select the antenna of the at least two antennas of the first device having the highest coupling with respect to at least one antenna of the second device, particularly so as to maintain a stable radio communication for different spatial orientations of the first and the second device with respect to each other.

Further, according to an embodiment of the system, the at least two antennas of the first device each comprise an antenna polarization, wherein said antenna polarizations are different, particularly orthogonal with respect to each other.

Further, according to an embodiment of the system, the at least two antennas of the first device are arranged at different locations.

Furthermore, according to an embodiment of the system, the first device is configured to control the at least two antennas of the first device in a phase-controlled manner in order to increase the coupling between said at least two antennas of the first device and the at least one antenna of the second device. By using a phased controlled antenna excitation the effective antenna gain can be improved leading to lower antenna coupling loss between the two devices.

However, alternatively, it is also possible that the first device is the external device that can, e.g., be configured to receive radio signals from a second device or to transmit radio signals to the second device. Here, the external device comprises the sensor and at least two antennas. Further, in this case, according to an embodiment, the second device can be a medical device configured to be implanted into a patient or to be arranged on the patient. For instance, the second device can be configured to transmit radio signals with position information regarding the position of the second device and/or the position of the patient to the external device. The signals are received by the external device, whereupon the external device selects one of the at least two antennas for transmitting radio signals to the second device. The antenna is selected depending on the position information.

Further, according to an embodiment, both devices, i.e., the first and the second device, can each comprise a sensor for measuring the respective spatial orientations as well as at least two antennas, wherein particularly the respective device is configured to select or control its antennas based on the output signal of the respective sensor, and particularly also depending on the output signal of the respective other sensor.

Particularly, according to an embodiment, the system (e.g., the first device) can be configured to also use said output signal of the sensor of the second device to select one of said antennas of the first device or to control said antennas of the first device for receiving a radio signal and/or for transmitting a radio signal (see also above).

Particularly, according to an embodiment, the present disclosure can be applied to all kind of (e.g., medical) devices. Particularly, the implantable medical device is a cardiac pacemaker, a neuro stimulating device or a monitoring device that records patient data and/or transmits patient data to the external device.

Furthermore, in case of a medical device that is configured to be arranged on the patient (from the outside), the medical device can be formed as a chest belt or may comprise a chest belt, wherein said chest belt is configured to be fastened to a chest of the patient. Here, particularly, said at least two antennas can be arranged at different locations along the chest belt and/or may comprise different, particularly orthogonal polarizations. Such a medical device having a chest belt can be a monitoring device, e.g., for sensing an electrocardiogram or other physiological data of the patient.

Moreover, the medical device can include a patch system which is attachable to the patient. According to an embodiment, the medical device can be worn by the patient in form of a belt, a watch, eyeglasses, a headband or any clothing item.

Further, according to an embodiment, the external device can be configured to control the first device and/or to communicate with the first device, particularly via radio signals. Particularly, the external device can be or comprise a remote control and/or data storage for storing patient data transmitted by the (e.g., implantable) medical device.

The respective antenna of the first and/or second device can be a dipole antenna or a monopole antenna. Furthermore, in all embodiments described herein, the respective external device can be a mobile device, particularly a hand-held device, or any stationary device, e.g., a wall mounted devices/antennas.

Further features, aspects, objects, advantages, and possible applications of the present invention will become apparent from a study of the exemplary embodiments and examples described below, in combination with the Figure, and the appended claims.

### DESCRIPTION OF THE DRAWINGS

Further features, advantages and embodiments will be described in the following with reference to the Figures, wherein:

FIG. 1 shows a coupling of two antennas, e.g., a transmitting antenna and a receiving antenna, for two different orientations of the antennas with respect to each other;

FIG. 2 shows an embodiment of a system comprising a first device in the form of an implantable medical device (e.g., a cardiac pacemaker) and an external second device, wherein the implantable medical device comprises at least two antennas and a sensor for sensing a spatial orientation of the implantable medical device;

FIG. 3 shows a further embodiment of a system comprising a first device in the form of a medical device having a chest belt or patch to be arranged on the patient (e.g., on the skin of the patient) and an external second device, wherein the medical device comprises at least two antennas and a sensor for sensing a spatial orientation of the medical device; and

FIG. 4 shows a variant of the embodiments shown in FIGS. 2 and 3, wherein here also the external second device comprises a sensor for sensing a spatial orientation of the external device and at least two antennas for sending radio signals to the medical device or for receiving radio signals from the medical device.

### DETAILED DESCRIPTION

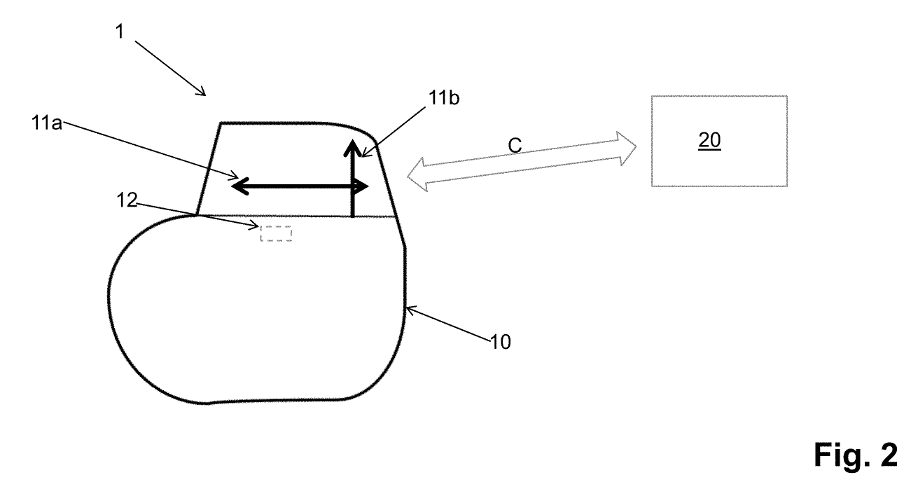

According to an embodiment of a system as shown in FIG. 2, the system 1 comprises at least a first device 10 having at least two antennas 11a, 11b for transmitting and/or receiving a radio signal C (e.g., to a second device 20 of the system), wherein the first device 10 comprises a sensor 12 that is configured to generate an output signal indicative of a spatial orientation of the first device 10 (e.g., with respect to some reference direction or with respect to the second device 20), wherein the first device 10 is configured to use the output signal to select one of said antennas 11a, 11b or to control said antennas 11a, 11b for receiving a radio signal C (e.g., from said second device 20) and/or for transmitting a radio signal C (e.g., to said second device 20).

Particularly, the first device 10 can be an implantable medical device 10 such as a cardiac pacemaker 10. According to an embodiment, an antenna 11a of said two antennas 11a, 11b can be a dipole antenna 11a whereas the other antenna 11b can be a monopole antenna. The antennas 11a, 11b particularly comprise different, particularly orthogonal polarizations as indicated in FIG. 2.

Thus, using the output signal of the sensor 12, the first device 10 is configured to use the antenna 11a or 11b that—due to its spatial orientation—comprises a higher coupling with an antenna of the external second device 20 which can be a remote control device and/or a device for recording data provided by the medical implant 10.

Alternatively, depending on said output signal, the first device 10 may also be configured to control the at least two antennas 11a, 11b of the first device 10 in a phase-controlled manner in order to increase a coupling between said at least two antennas 11a, 11b of the first device 10 and at least one antenna of the second device 20.

Thus, in the embodiment shown in FIG. 2 the implantable medical device 10 particularly uses the sensor 12 to determine the current spatial orientation with respect to the external transmitting or receiving device 20. In case the spatial position of the implantable medical device 10 changes relative to the external device 20, the most suitable antenna 11a, 11b is selected based on the output signal of the sensor 12 which is indicative of said spatial orientation. Here, particularly, the notion most suitable can mean that the antenna 11a, 11b having the best transmitting and/or receiving characteristics with respect to the external device 20.

In some cases, the orientation of the external device is defined by its design or by the operating manual and is therefore known. If this is not the case, the external device 20 has to estimate it's spatial orientation with its sensor 22 and transmit this information back to the implantable medical device.

According to an embodiment, the external device 20 is a stationary device with unchanging position and orientation in a room. The position of the stationary device serves as reference for implantable device 10 in order to choose the most suitable antenna 11a or 11b for data transmission with the external device 20. According to another embodiment, the external device 20 is not stationary, i.e., the position of the external device 20 is variable. In this case, the external device 20 and the implantable device 10 exchange data regarding their position and/or orientation in order to select the most suitable antenna 11a, 11b prior to radio signal data transmission.

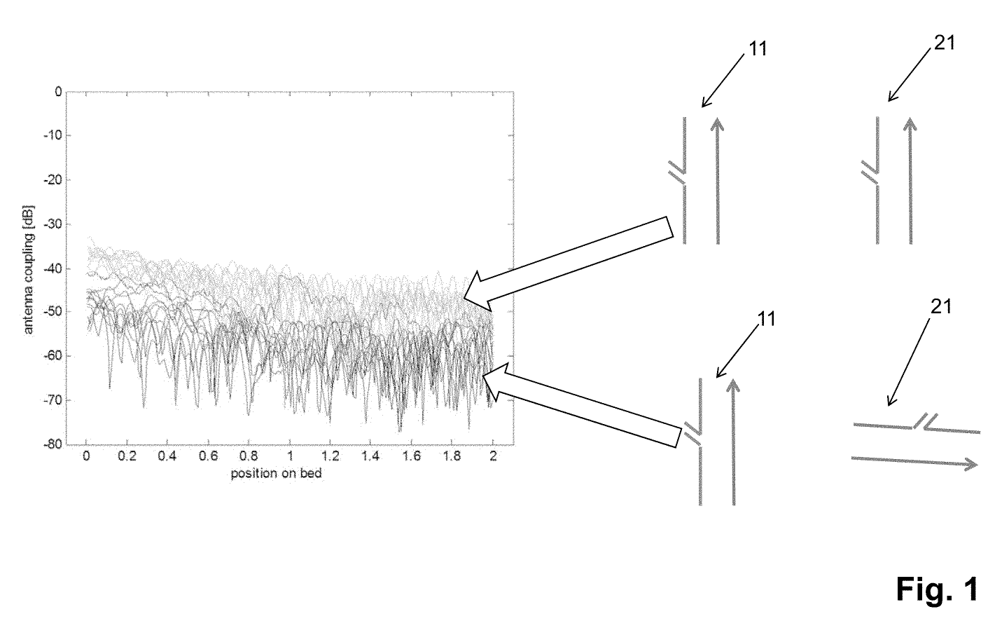

The dependence of the coupling between two antennas, say a transmitting antenna 11 and a receiving antenna 21, that is utilized by the present disclosure is demonstrated in FIG. 1, which indicates that the coupling between said two antennas 11, 21 as shown in the graph on the left-hand side of FIG. 1 depends on the spatial orientations of the two antennas 11, 21 with respect to one another. In this regard, the upper part of the right-hand side of FIG. 1 shows the two antennas 11, 21 having aligned polarizations, which yields a better coupling than in the case where the two polarizations are oriented in different directions (lower part of the right-hand side of FIG. 1).

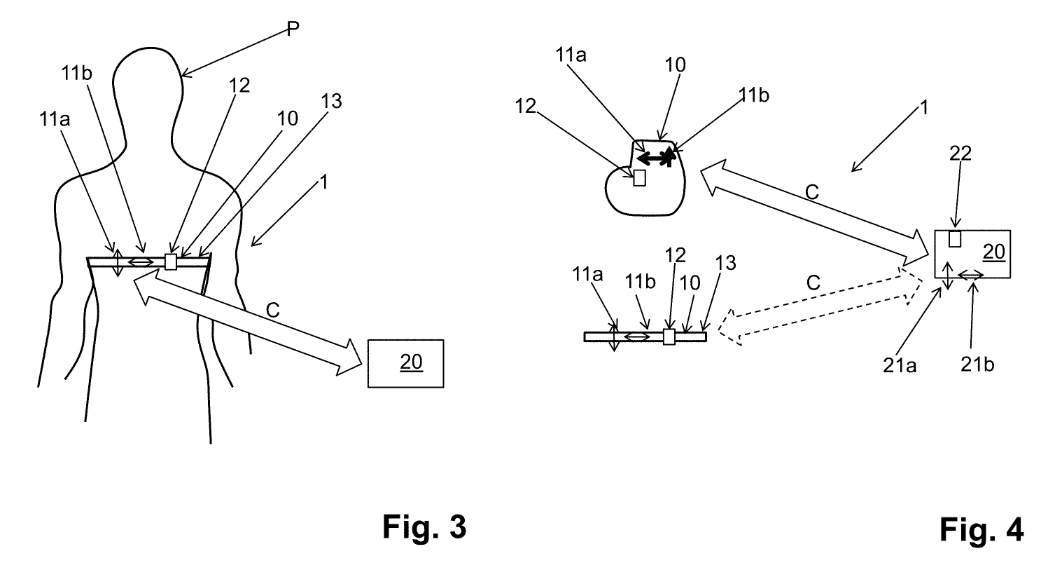

According to a further embodiment shown in FIG. 3, the first device 10 can also be a medical device that is configured to be arranged on the patient P (e.g., on the skin of the patient). Such devices 10 are often referred to as patches. Here the first device 10 may comprise a chest belt 13 that can be fastened to the chest of the patient P. Also here, the medical device (e.g., patch) 10 comprises at least two independent antennas 11a, 11b as well as a sensor 12 configured to measure a spatial orientation of the medical device 10 with respect to, e.g., an external (second) device 20 that may be used to record data transmitted by the first device 10 via radio signals C. In case the spatial orientation of the patch 10 now changes relative to the external device 20, again the most suitable antenna 11a, 11b of the first device 10 is selected—as before—based on the output signal of the sensor 12 which is indicative of said spatial orientation of the first device 10.

Finally, FIG. 4 shows further embodiments, wherein additionally—besides determining the spatial orientation of the respective medical device 10—the external (e.g., mobile) device 20 comprises a sensor 22 that is configured to determine the spatial orientation of the external device 20, wherein a corresponding output signal of the sensor 22 that is indicative of the spatial orientation of the external device 20 is also used to select one of said antennas 11a, 11b of the medical device 10 or to control said antennas 11a, 11b. Alternatively, or in addition, said output signal of the sensor 22 of the second device 20 may be used to select an antenna 21a, 21b of a plurality of antennas of the external device 20 so as to improve a coupling between an active antenna 11a, 11b of the medical device 10 and an active antenna 21a, 21b of the external device 20.

In the embodiments described above, the respective device 10, 20 that comprises at least two antennas 11a, 11b or 21a, 21b may also comprise more than two antennas between which the respective device may choose, e.g., for improving radio communication based on a spatial orientation of the respective device 10, 20.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

I/We claim:

1. A system, comprising:
a first device having at least two antennas for transmitting and/or receiving a radio signal, wherein the first device is a medical device configured to be implanted into a patient, wherein the first device comprises a sensor that is configured to generate an output signal indicative of a spatial orientation of the first device, and wherein the first device is configured to use the output signal to select one of said antennas or to control said antennas for receiving a radio signal and/or for transmitting a radio signal, and
a second device having at least one antenna, wherein the second device is configured to transmit a radio signal to the first device and/or to receive a radio signal from the first device, wherein the second device is an external device.

2. The system according to claim 1, wherein the first device is configured to select the antenna of the at least two antennas having the highest coupling with respect to the at least one antenna of the second device.

3. The system according to claim 1, wherein the at least two antennas of the first device each comprise an antenna polarization, wherein said antenna polarizations are different with respect to each other.

4. The system according to claim 3, wherein said antenna polarizations are orthogonal with respect to each other.

5. The system according to claim 1, wherein the at least two antennas of the first device are arranged at different locations.

6. The system according to claim 1, wherein the first device is configured to control the at least two antennas of the first device in a phase-controlled manner in order to increase a coupling between said at least two antennas of the first device and the at least one antenna of the second device.

7. The system according to claim 1, wherein the second device comprises a sensor that is configured to generate an output signal indicative of a spatial orientation of the second device.

8. The system according to claim 7, wherein the second device comprises at least two antennas, wherein the second device is configured to use said output signal of the sensor of the second device to select an antenna of the at least two antennas of the second device for receiving a radio signal from the first device and/or for transmitting a radio signal to the first device.

9. The system according to claim 7, wherein the system is configured to use said output signal of the sensor of the second device to select one of said antennas of the first device or to control said antennas of the first device for receiving a radio signal and/or for transmitting a radio signal.
